(19) United States
(12) Patent Application Publication
(10) Pub. No.: US 2020/0094252 A1
(43) Pub. Date: Mar. 26, 2020

(54) MICROFLUIDIC CHIP FOR SEPARATING AND DETECTING WHOLE BLOOD SAMPLE AND DETECTION METHOD THEREOF

(71) Applicant: MEDICAL SYSTEM BIOTECHNOLOGY CO., LTD., NINGBO (CN)

(72) Inventors: QIANG CHEN, NINGBO (CN); BINGDE ZOU, NINGBO (CN); JIHUA ZOU, NINGBO (CN); TENG TANG, NINGBO (CN); ANJI SUN, NINGBO (CN)

(73) Assignee: MEDICAL SYSTEM BIOTECHNOLOGY CO., LTD.

(21) Appl. No.: 16/594,816

(22) Filed: Oct. 7, 2019

Related U.S. Application Data
(63) Continuation of application No. PCT/CN2017/108527, filed on Oct. 31, 2017.

(30) Foreign Application Priority Data
Apr. 6, 2017 (CN) 201710219876.9

(51) Int. Cl. B01L 3/00; G01N 33/543

(52) U.S. Cl. CPC B01L 3/502761; G01N 33/54366; B01L 2300/087; B01L 2200/16; B01L 2300/0851; B01L 2200/0652; B01L 2200/10; B01L 2300/0816; B01L 2400/0487; B01L 2300/0883

(57) ABSTRACT

The present disclosure discloses a microfluidic chip for separating and detecting whole blood sample. It has a chip body and a sample channel on the said chip body. The sample channel has a sample-feeding area, a sinking area, a mixing area, a testing area and a waste liquor area connected in sequence. The microfluidic chip integrates separating and testing of plasma in whole blood into a whole, free from a complicated whole blood sample pre-treatment process, and rapidly detect single or multiple proteins or other indicators in whole blood in a quantified manner.

### CROSS-REFERENCE TO RELATED APPLICATIONS

This application is a continuation of International Patent Application No. PCT/CN2017/108527 with a filing date of Oct. 31, 2017, designating the United States, now pending, and further claims priority to Chinese Patent Application No. 201710219876.9 with a filing date of Apr. 6, 2017. The content of the aforementioned applications, including any intervening amendments thereto, are incorporated herein by reference.

### TECHNICAL FIELD

The present disclosure relates to the technical field of fluid sample testing, in particular to a microfluidic chip for separating and detecting whole blood sample and detection method thereof.

### BACKGROUND

It is a primary medical program to analyze components and their contents of blood in modern medical testing. Whole blood is comprised by liquid plasma and hemocyte, but in view of a great interference on chromatographic analysis from hemocyte or hemoglobin, it often needs to separate plasmas from a blood sample for biochemical or immunodiagnostic analysis. At present, there are two common methods for plasma separation in clinic, i.e., a centrifugation method and a filtering method, which however both have respective disadvantages, to be specific, the centrifugation method involves large-sized equipment and is complicate to operate, while the filtering method has low separation efficiency and easily suffers from sample pollution.

Currently, the POCT (Point of Care Testing) technology has been applied more and more widely. As it requires rapid detection analysis on the sampling site, complicate and time-consuming treatment of the sample in a lab is omitted, detection equipment and reagents are convenient to carry, and the operation is simple. In recent years, the micro total analysis systems (uTAS) have raised considerable concern due to its micromation, integration and intelligentization, especially for the sake of its advantages of rapid analysis speed and low sample consumption, thereby providing a better detection platform for medical testing. The microfluidic chip, as the core technology of the uTAS, has the capability of integrating such operations as sample separation, mixing, reaction, testing and the like on several square centimeters, and thus is more applicable to the POCT. Therefore, how to achieve plasma separation on the microfluidic chip and quantified detection of its contents is the technical problem that urgently needs to be addressed in the field.

### SUMMARY

The technical problem to be solved by the present disclosure is to provide a microfluidic chip for separating and detecting whole blood sample, which integrates separating and testing of plasma in whole blood into a whole, free from a complicated whole blood sample pre-treatment process, and rapidly detect single or multiple proteins or other indicators in whole blood in a quantified manner.

The technical solution is to provide a microfluidic chip for separating and detecting whole blood sample, comprising a chip body on which a sample channel is provided; the sample channel comprises a sample-feeding area, a sinking area, a mixing area, a testing area and a waste liquor area connected in sequence; the sinking area comprises a sample-feeding portion and a sinking portion, wherein one end of the sample-feeding portion is connected with the sample-feeding area, while its other end is connected with one end of the sinking portion; the ratio of the largest widths between the sinking portion and the sample-feeding portion is 2-10; the sinking portion is wide in the middle and narrow at two sides; the front and rear lateral walls of two end portions of the sinking portion are both inclined surfaces; extending lines of the front and rear lateral walls intersect to form an included angle; the front and rear lateral walls of the middle of the sinking portion are parallel surfaces that are parallel to each other.

With the above structure, the microfluidic chip for separating and detecting whole blood sample has the following advantages as compared to the prior art:

Since the ratio of the largest widths between the sinking portion and the sample-feeding portion of the sinking area is 2-10 used in the microfluidic chip for separating and detecting whole blood sample in the present disclosure, a sample is controlled at a moderate speed after entering the sinking area, meanwhile, generated air bubbles are moderate in size, leading to a good plasma and hemocyte separation effect. If the ratio of the largest widths between the sinking portion and the sample-feeding portion is less than 2, oversized speed variation of the sample after entering the sinking area is adverse to hemocyte settlement, further oversized air bubbles generated may cause hemocyte to be remixed into separated plasma to lead to separation failure. If the ratio of the largest widths between the sinking portion and the sample-feeding portion is more than 10, generated air bubbles become tiny and dispersive, therefore, separation of hemocyte and plasma becomes impossible, resulting in incomplete separation. As an improvement, the sample-feeding portion is a straight tube. The sinking portion is wide in middle but narrow in two sides. Such the structure will give the sample a large speed variation after entering the sinking area, contributive to separation of hemocyte and plasma.

As an improvement, the sample-feeding portion is a straight tube. The sinking portion is wide in the middle and narrow at two sides. This structure will provide a large speed variation for the sample after entering the sinking area, contributive to separation of hemocyte and plasma.

As an improvement, the sample-feeding area, the sinking area, the mixing area, the testing area and the waste liquor area of the sample channel are accordant in depth. By adopting the above structure, the chip manufacture process becomes relatively simple, resulting in low manufacture cost.

As an improvement, the depth of the sample-feeding area of the sample channel equals to that of the sinking area, and equals to a first depth; the depth of the said mixing area of the sample channel equals to that of the testing area, equals to that of waste liquor area, and equals to a second depth; the said first depth is larger than the second depth; the said bottom wall of the sinking area levels with the bottom wall of the mixing area. With the above structure, the depth of the mixing area is less deeper than the depth of the sinking area, and the flow rate of blood plasma in the mixing area is sped up so as to produce a good mixing effect of blood plasma and reactants.

As an improvement, the chip body also comprises a cleaning solution storage area, and an outlet of a cleaning solution tube of the cleaning solution storage area is connected between the mixing area and the testing area. By adopting the above structure, after a blood plasma mixture completely flows through the testing area, the cleaning solution tube is opened, and then a cleaning solution in the cleaning solution storage area flows into the testing area to flush uncombined reactants into the waste liquor area, thus achieving a good testing effect.

As an improvement, the cleaning solution storage area comprises a cleaning solution tank isolated from air. An inlet of the cleaning solution tube is communicated with the cleaning solution tank that is internally provided with a cleaning solution cup filled with a cleaning solution. The tank bottom of the cleaning solution tank is provided with a piercing piece for piercing through the bottom wall of the cleaning solution cup. After adopting the above structure, in case of using the cleaning solution, the cleaning solution cup is pressed down mechanically or artificially to enable the piercing piece to pierce through the bottom wall of the cleaning solution cup such that the cleaning solution therein flows into the cleaning solution tank. A seal structure of the cleaning solution tank is damaged mechanically or artificially so as to communicate the cleaning solution tank with air. Afterwards, under the effect of a pump, the cleaning solution is pumped into the testing area which is simple in structure and convenient in use.

As an improvement, the chip body comprises a cover plate and a bottom plate. The sample-feeding area, the sinking area, the mixing area, the testing area and the waste liquor area are all positioned on the said cover plate. The testing area has an opening at the bottom. The bottom plate is connected to the lower side of the cover plate. The bottom plate is provided with test strips at the locations corresponding to the opening. By adopting the above structure, the chip structure is simple, and the manufacture is convenient.

As an improvement, the mixing area is internally provided with a zigzag-shaped channel or a S-shaped channel or a W-shaped channel. By adopting the above structure, the blood plasma and reactant mixing effect is relatively better.

The other technical problem to be settled by the present disclosure is to provide a detection method of the microfluidic chip for separating and detecting whole blood sample, which integrates the separating and testing of plasma in whole blood into a whole, free from a complicated whole blood sample pre-treatment process, and rapidly detect single or multiple proteins or other indicators in whole blood in a quantified manner.

The technical solution adopted to resolve the above technical problem provides a detection method of the microfluidic chip for separating and detecting whole blood sample, comprising the following steps:

Step 1, connecting a quantified sampling tube to the sample-feeding area of the microfluidic chip, contacting the quantified sampling tube with a whole blood sample, and completing quantified sampling of the whole blood sample under the capillary action;

Step 2, applying negative-pressure drive to the port of the waste liquor area of the microfluidic chip, mixing and reacting the sample after entering the sinking area of the microfluidic chip with a settling promoter that volatilized to dryness in the sinking area, rapidly settling the hemocyte in the sample, after a period of times, allowing air to enter from the sampling tube to isolate the hemocyte from plasma, wherein the plasma flows into the mixing area of the microfluidic chip, while the hemocyte totally retains in the sinking area of the microfluidic chip;

Step 3, re-dissolving the plasma with a fluorescent primary antibody that volatilizes and dry in the mixing area, uniformly mixing and reacting under the cooperation of the channel structure in the mixing area to form an antigen-immunofluorescent primary antibody compound that then enters the testing area of the microfluidic chip;

Step 4, subjecting the antigen-immunofluorescent primary antibody compound to have a specific reaction with a secondary antibody fixed on the test strips of the microfluidic chip to form a secondary antibody-antigen-fluorescent primary antibody sandwiched structure;

Step 5, after a blood plasma mixture totally flows through the testing area, opening a cleaning solution branch channel of the microfluidic chip, and allowing the cleaning solution to flow into the testing area to flush uncombined fluorescent primary antibody into the waste liquor area;

Step 6, by detecting fluorescence intensity of the test strips, achieving quantified detection of an antigen in the sample.

By adopting the above steps, the detection method of the microfluidic chip for separating and detecting whole blood sample has the following advantages as compared to the prior art:

In the detection method of the microfluidic chip for separating and detecting whole blood sample in the present disclosure, a whole blood sample is sucked into the microfluidic chip by negative pressure, hemocyte and blood plasma are isolated by air in the sinking area, the plasma flows in the mixing area and then re-dissolves with a fluorescent primary antibody in the mixing area to form an antigen-immunofluorescent primary antibody compound that enters the testing area of the microfluidic chip afterwards, the antigen-immunofluorescent primary antibody compound has a specific reaction with a secondary antibody on the test strips fixed on the microfluidic chip to form a secondary antibody-antigen-fluorescent primary antibody sandwiched structure, a cleaning solution branch channel of the microfluidic chip is opened, and the cleaning solution flows into the testing area to flush uncombined fluorescent primary antibody into the waste liquor area, therefore, a detection method is realized with a better testing effect.

As an improvement, the sinking area comprises a sample-feeding portion and a sinking portion, wherein one end of the sample-feeding portion is connected with the sample-feeding area, while its other end is connected with one end of the sinking portion; the ratio of the largest widths between the sinking portion and the sample-feeding portion is 2-10; the sinking portion is wide in the middle and narrow at two sides; the front and rear lateral walls of two end portions of the sinking portion are both inclined surfaces; the extending lines of the front and rear lateral walls intersect to form an included angle; front and rear lateral walls of the middle of the sinking portion present parallel surfaces that are parallel to each other. By adopting the above structure, the sample is controlled at a moderate speed after entering the sinking area, meanwhile, generated air bubbles are moderate in size, therefore, a plasma and hemocyte separation effect is good. If the ratio of the largest widths between the sinking portion and the sample-feeding portion is less than 2, undersized speed variation of the sample after entering the sinking area is adverse to hemocyte settlement, further oversized air bubbles generated may cause the hemocyte to be remixed into separated plasma to lead to separation failure. If the ratio of the largest widths between the sinking portion and the sample-feeding portion is more than 10, generated air bubbles are tiny and dispersive, such that separation of hemocyte and plasma becomes impossible so as to cause incomplete separation.

### BRIEF DESCRIPTION OF THE DRAWINGS

FIG. 1 is a schematic diagram of an exploded structure of a microfluidic chip for separating and detecting whole blood sample of the present disclosure.

FIG. 2 is a schematic structural diagram of a channel of the microfluidic chip for separating and detecting whole blood sample of the present disclosure.

FIG. 3 is a schematic structural diagram of a cleaning solution cup and a piercing piece of the microfluidic chip for separating and detecting whole blood sample of the present disclosure.

FIG. 4 is a diagram depicting the separation process of the microfluidic chip for separating and detecting whole blood sample.

FIG. 5 is a comparative diagram of the separation effects between the microfluidic chip for separating and detecting whole blood sample of the present disclosure and a centrifugal machine.

Reference numerals: 1 sample-feeding area; 2 sinking area; 2.1 sample-feeding portion; 2.2 sinking portion; 3 mixing area; 4 testing area; 5 waste liquor area; 6 cover plate; 7 opening; 8 bottom plate; 9 test strip; 11 cleaning solution storage area; 12 cleaning solution tube; 13 cleaning solution tank; 14 cleaning solution cup; 15 piercing piece; 16 zigzag-shaped channel; 17 quantified sampling tube.

### EMBODIMENTS

The present disclosure will be further explained hereafter by referring to the following embodiments and appended drawings.

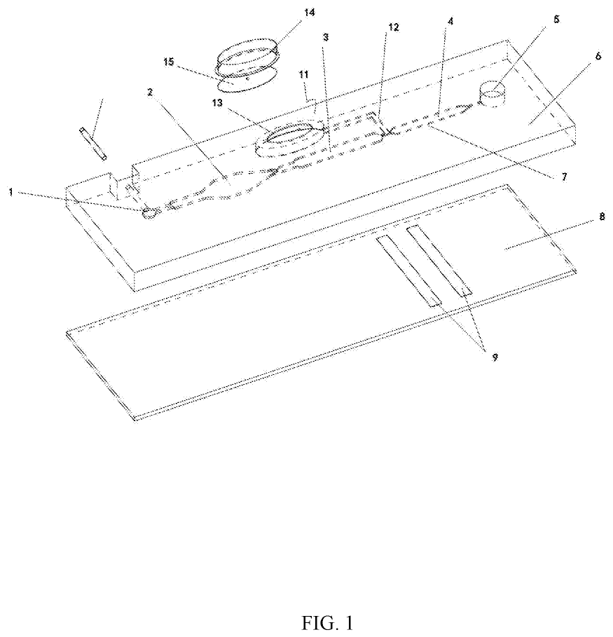

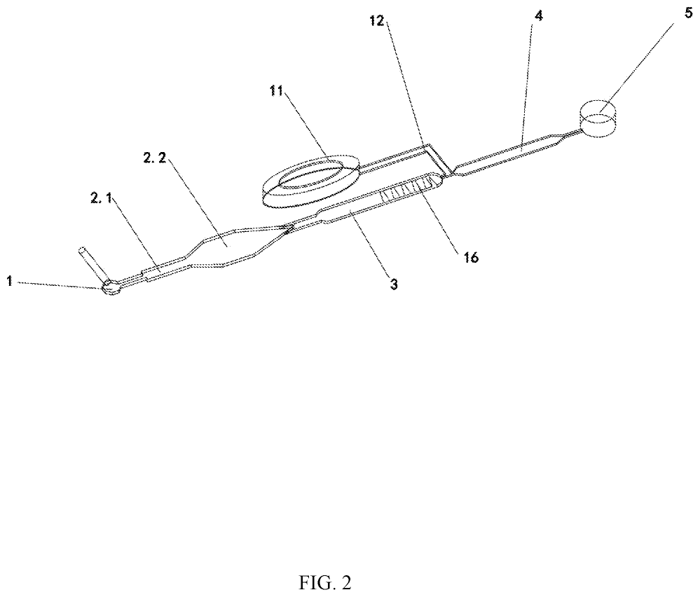

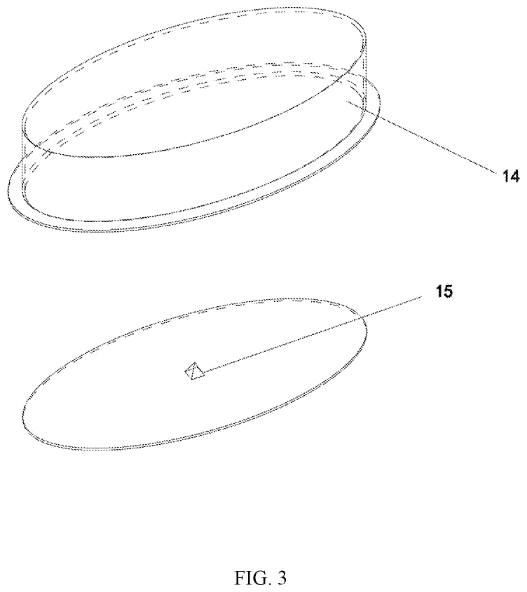

As shown in FIG. 1-FIG. 3, a microfluidic chip for separating and detecting whole blood sample, comprises a chip body on which a sample channel is provided. The sample channel comprises a sample-feeding area 1, a sinking area 2, a mixing area 3, a testing area 4 and a waste liquor area 5 connected in sequence. In this embodiment, the chip body comprises a cover plate 6 and a bottom plate 8. The sample-feeding area 1, the sinking area 2, the mixing area 3, the testing area 4 and the waste liquor area 5 are all positioned on the cover plate 6. The testing area 4 has an opening 7 at the bottom. The bottom plate 8 is connected to the lower side of the cover plate 6. The bottom plate 8 is provided with two parallel test strips 9 at the locations corresponding to the opening 7. The testing area 4 is arranged along the length direction of the cover plate 6. The test strips 9 are arranged along the width direction of the bottom plate 8. The bottom of the cover plate 6 is provided with a groove to receive the test strips 9. After the cover plate 6 and the bottom plate 8 are assembled together, the test strips 9 are received in the groove. In this embodiment, the test strip 9 is 10-30 mm in length and 1-10 mm in width.

The microchannel and microstructure of the cover plate 6 are manufactured by a cast molding process, a hot pressing process, a laser etching process, a soft lithography process or the like. In this embodiment of the present disclosure, the microfluidic chip is preferably manufactured by the soft lithography process, that is, by taking a polished silicon wafer as a substrate material and an SU-8 photoresist as a mask layer, carrying out exposure, development and other steps to manufacture a mold of the cover plate; pouring PDMS (Sylgard 184) on the mold, heating and curing, and peeling off from the mold to obtain a PDMS chip; and punching at the sampling port and the waste liquor area to produce the cover plate.

The sinking area 2 comprises a sample-feeding portion 2.1 and a sinking portion 2.2, wherein one end of the sample-feeding portion is connected with the sample-feeding area, while its other end is connected with one end of the sinking portion. The ratio of the largest width a of the sinking portion and the largest width b of the sample-feeding portion is 2-10. In this embodiment, the ratio of the largest width a of the sinking portion and the largest width b of the sample-feeding portion is 3.125. The effect is relatively good as well if the ratio falls into the scope of 3-3.5. The sinking area is 1-50 mm in length and 0.5-10 mm in width.

The sample-feeding portion 2.1 is a straight tube. The sinking portion 2.2 is wide in the middle and narrow at two sides. The front and rear lateral walls of two end portions of the sinking portion 2.2 are both inclined surfaces. The extending lines of the front and rear lateral walls intersect to form an included angle. The front and rear lateral walls of the middle of the sinking portion 2.2 are parallel surfaces that are parallel to each other. The front and rear lateral walls of each end portion of the sinking portion 2.2 are equal in length. The front and rear lateral walls of each end portion of the sinking portion 2.2 form equal angles with the sample-feeding portion 2.1 respectively.

The chip body also comprises a cleaning solution storage area 11. An outlet of a cleaning solution tube 12 of the cleaning solution storage area 11 is connected between the mixing area 3 and the testing area 4. The cleaning solution storage area 11 comprises a cleaning solution tank 13 isolated from air, An inlet of the cleaning solution tube 12 is communicated with the cleaning solution tank 13 having a top opening. The top opening of the cleaning solution tank 13 is provided with an isolated film. In case of using the cleaning solution, the isolated film is pierced through mechanically or artificially so as to communicate the cleaning solution tank 13 with air. The cleaning solution tank 13 is internally provided with a cleaning solution cup 14 filled with a cleaning solution. The tank bottom of the cleaning solution tank 13 is provided with a piercing piece 15 for piercing through the bottom wall of the cleaning solution cup. The bottom of the cleaning solution cup 14 is made of a thin film that is easily pierced through by the piercing piece 15.

The mixing area 3 is internally provided with a zigzag-shaped channel 16 or a S-shaped channel or a W-shaped channel. The length of the zigzag-shaped channel 16 or S-shaped channel or W-shaped channel is less than the length of the mixing area 3. The zigzag-shaped channel or S-shaped channel or W-shaped channel is arranged at one end of the mixing area 3 close to the testing area 4. The mixing area 3 is 0.5-5 mm in width.

The sample-feeding area 1, the sinking area 2, the mixing area 3, the testing area 4 and the waste liquor area 5 of the sample channel are accordant in depth, and the said depth is 0.5-10 mm.

In another embodiment, the depth of the sample-feeding area 1 of the sample channel equals to that of the sinking area 2, and equals to a first depth; the depth of the mixing area 3 of the sample channel equals to that of the testing area 4, equals to that of waste liquor area 5, and equals to a second depth; the first depth is larger than the second depth; the bottom wall of the sinking area 2 levels with the bottom wall of the mixing area 3. The first depth is 0.5-10 mm. The second depth is 10-300 um.

The microfluidic chip for separating and detecting whole blood sample also comprises a quantified sampling tube 17 that is a capillary glass tube of a certain volume. When in use, the quantified sampling tube 17 is connected to the sample-feeding area 1 of the microfluidic chip, and quantified sampling of the whole blood sample is completed under the effect of the quantified sampling tube 17.

Before using the microfluidic chip for separating and detecting whole blood sample, a settling promoter is volatilized to dryness in the sinking area 2 in advance, that is, the settling promoter is placed in the sinking area 2 in advance, and stood for a period of time to volatilize water moisture therein; a fluorescently-labeled primary antibody reagent volatilizes dry in the mixing area 3 in advance, that is, the fluorescently-labeled primary antibody reagent is placed in the mixing area 3 in advance and stood for a period of time to volatilize water moisture therein; a secondary antibody is fixed on the test strips in the testing area 4 in advance, to be specific, a 2 mg/mL coated antibody and Rabbit IgG are coated on T-line and C-line sites of an aldehyde substrate separately, fixed for 2 h at the temperature of 37° C., and cleaned three times with a cleaning solution (pH7.4 10 mM PBS+0.05% Tween 20), and once with pure water; the aldehyde substrate is immersed into a blocking solution (pH7.4 10 mM PBS+0.3755% Gly+1% BSA+0.1% NaN3), closed for 2 h at room temperature, cleaned three times with the cleaning solution and then once with clean water, and stood overnight in a low-humidity environment.

When in use, a negative pressure pump or peristaltic pump is externally connected to the waste liquor area 5 of the microfluidic chip, and a sample is driven by air pressure difference to flow through the whole chip.

The detection method of the microfluidic chip for separating and detecting whole blood sample, comprises the following steps:

Step 1, contacting a quantified sampling tube with a whole blood sample, and completing quantified sampling of the whole blood sample under the capillary action;

Step 2, placing a microfluidic chip into an auxiliary instrument, applying negative-pressure drive to the port of the waste liquor area, feeding the sample into the sinking area to be mixed and reacted with a settling promoter that volatilized to dryness, rapidly settling the hemocyte in the sample, after a period of time, allowing air to enter from a sampling tube to isolate the hemocyte from plasma, enabling the plasma to flow into the mixing area, and totally retaining the hemocyte in the sinking area;

Step 3, re-dissolving the plasma with a fluorescent primary antibody that volatilized to dryness in the mixing area, by means of the channel structure in the mixing area, uniformly mixing and reacting to form an antigen-immunofluorescent primary antibody compound that then enters the testing area;

Step 4, in the testing area, subjecting the antigen-immunofluorescent primary antibody compound to have a specific reaction with a secondary antibody fixed on the test strips to form a secondary antibody-antigen-fluorescent primary antibody sandwiched structure;

Step 5, after a blood plasma mixture totally flows through the testing area, opening a cleaning solution branch channel, allowing the cleaning solution to flow into the testing area to flush uncombined fluorescent primary antibody into the waste liquor area;

Step 6, by detecting fluorescence intensity, implementing quantified detection of an antigen in the sample.

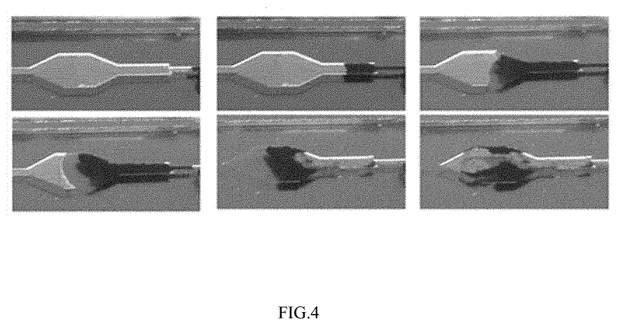

FIG. 4 is a diagram depicting the separation process of the microfluidic chip for separating and detecting whole blood sample. Blood flows into the sinking area under the negative pressure drive, after entry into a wide sinking portion from a narrow straight tube, flow rate of blood rapidly slows down, with the help of the setting promoter, hemocyte agglomeration settles under the effect of gravity, and plasma is separated out at the front of the whole fluid. After the blood sample totally enters the sinking portion, the air enters so as to completely isolate the hemocyte from the plasma, the plasma unceasingly flows for subsequent reactions, but the hemocyte subsides in the sinking area.

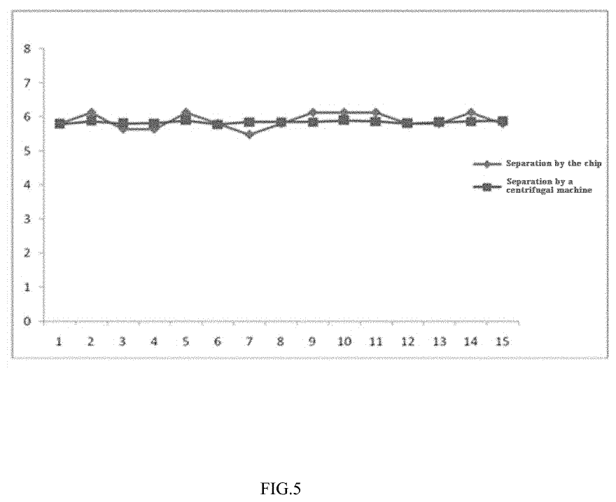

FIG. 5 is a comparative diagram of the separation effects between the microfluidic chip for separating and detecting whole blood sample of the present disclosure and a centrifugal machine. This diagram shows the statistical data that are acquired by carrying out repeated testing on the same blood sample for several times. The same blood sample is subjected to plasma separation respectively by this chip of the present disclosure and a traditional centrifugal machine, separated plasma volumes are measured and testing is performed for 15 times. The data indicate that the chip of the present disclosure has the stability relatively closing to that of a large-scale centrifugal machine used in the traditional centrifugal machine separation method.

We claim:

1. A microfluidic chip for separating and detecting whole blood sample, wherein comprises a chip body, and a sample channel on the said chip body; the said sample channel comprises a sample-feeding area (1), a sinking area (2), a mixing area (3), a testing area (4) and a waste liquor area (5) connected in sequence; the sinking area (2) comprises a sample-feeding portion (2.1) and a sinking portion (2.2); one end of the sample-feeding portion (2.1) is connected with the sample-feeding area (1), and the other end of the sample-feeding portion (2.1) is connected with one end of the sinking portion (2.2); the ratio of the largest width of the sinking portion (2.2) and the largest width of the sample-feeding portion (2.1) is 2-10; the sinking portion (2.2) is wide in the middle and narrow at two sides; the front and rear lateral walls of two end portions of the sinking portion (2.2) are both inclined surfaces; the extending lines of the front and rear lateral walls intersect to form an included angle; the front and rear lateral walls of the middle of the sinking portion (2.2) are parallel surfaces that are parallel to each other.

2. The microfluidic chip for separating and detecting whole blood sample of claim 1, wherein the sample-feeding portion (2.1) is a straight tube.

3. The microfluidic chip for separating and detecting whole blood sample of claim 1, wherein the sample-feeding area (1), the sinking area (2), the mixing area (3), the testing area (4) and the waste liquor area (5) of the sample channel are accordant in depth.

4. The microfluidic chip for separating and detecting whole blood sample of claim 1, wherein the depth of the sample-feeding area (1) of the sample channel equals to the depth of the sinking area (2), and equals to a first depth; the depth of the said mixing area (3) of the sample channel equals to the depth of the testing area (4), equals to the depth of waste liquor area (5), and equals to a second depth; the said first depth is larger than the second depth; the said bottom wall of the sinking area (2) levels with the bottom wall of the mixing area (3).

5. The microfluidic chip for separating and detecting whole blood sample of claim 1, wherein the chip body comprises a cleaning solution storage area (11); an outlet of a cleaning solution tube (12) of the cleaning solution storage area (11) is connected between the mixing area (3) and the testing area (4).

6. The microfluidic chip for separating and detecting whole blood sample of claim 5, wherein the cleaning solution storage area (11) comprises a cleaning solution tank (13) isolated from air; an inlet of the cleaning solution tube (12) is communicated with the cleaning solution tank (13); the cleaning solution tank (13) is internally provided with a cleaning solution cup (14) filled with a cleaning solution; the tank bottom of the cleaning solution tank (13) is provided with a piercing piece (15) for piercing through the bottom wall of the cleaning solution cup (14).

7. The microfluidic chip for separating and detecting whole blood sample of claim 1, wherein the chip body comprises a cover plate (6) and a bottom plate (8); the said sample-feeding area (1), the sinking area (2), the mixing area (3), the testing area (4) and the waste liquor area (5) are all positioned on the cover plate (6); the testing area (4) has an opening (7) at the bottom; the bottom plate (8) is connected to the lower side of the cover plate (6); the bottom plate (8) is provided with a test strip (9) at the location corresponding to the opening (7).

8. The microfluidic chip for separating and detecting whole blood sample of claim 1, wherein the mixing area (3) is internally provided with a zigzag-shaped channel (16) or a S-shaped channel or a W-shaped channel.

9. A detection method of the microfluidic chip for separating and detecting whole blood sample, comprises the following steps:
Step 1, connecting a quantified sampling tube (17) to the sample-feeding area of the microfluidic chip, contacting the quantified sampling tube (17) with a whole blood sample, and completing quantified sampling of the whole blood sample under the capillary action;
Step 2, applying negative-pressure drive to the port of the waste liquor area (5) of the microfluidic chip, mixing and reacting the sample after entering the sinking area (2) of the microfluidic chip with a settling promoter that volatilized to dryness in the sinking area (2), rapidly settling the hemocyte in the sample, after a period of times, allowing air to enter from the sampling tube (17) to isolate the hemocyte from plasma, wherein the plasma flows into the mixing area (3) of the microfluidic chip, while the hemocyte totally retains in the sinking area (2) of the microfluidic chip;
Step 3, re-dissolving the plasma with a fluorescent primary antibody that volatilizes and dry in the mixing area (3), uniformly mixing and reacting under the cooperation of the channel structure in the mixing area to form an antigen-immunofluorescent primary antibody compound that then enters the testing area (4) of the microfluidic chip;
Step 4, in the testing area (4), subjecting the antigen-immunofluorescent primary antibody compound to have a specific reaction with a secondary antibody fixed on the test strips (9) of the microfluidic chip to form a secondary antibody-antigen-fluorescent primary antibody sandwiched structure;
Step 5, after a blood plasma mixture totally flows through the testing area (4), opening a cleaning solution branch channel of the microfluidic chip, and allowing the cleaning solution to flow into the testing area (4) to flush uncombined fluorescent primary antibody into the waste liquor area (5);
Step 6, by detecting fluorescence intensity of the test strips (9), achieving quantified detection of an antigen in the sample.

10. The detection method of the microfluidic chip for separating and detecting whole blood sample of claim 9, wherein the sinking area (2) comprises a sample-feeding portion (2A) and a sinking portion (2.2); one end of the sample-feeding portion (2.1) is connected with the sample-feeding area (1), and the other end of the sample-feeding portion (2.1) is connected with one end of the sinking portion (2.2); the ratio of the largest width of the sinking portion (2.2) and the largest width of the sample-feeding portion (2.1) is 2-10; the sinking portion (2.2) is wide in the middle and narrow at two sides; the front and rear lateral walls of two end portions of the sinking portion (2.2) are both inclined surfaces; the extending lines of the front and rear lateral walls intersect to form an included angle; the front and rear lateral walls of the middle of the sinking portion (2.2) are parallel surfaces that are parallel to each other.
